(19) United States
(12) Patent Application Publication
(10) Pub. No.: US 2026/0315508 A1
(43) Pub. Date: Oct. 15, 2026

(54) Large-leaf yellow tea oligosaccharides with GLP-1 agonist activity and use thereof

(71) Applicant: Anhui Agricultural University, Hefei City (CN)

(72) Inventors: Hongyan WANG, Hefei City (CN); Hai XIE, Hefei City (CN); Daxiang LI, Hefei City (CN); Yijun WANG, Hefei City (CN); Zhongwen XIE, Hefei City (CN); Huifang GE, Hefei City (CN); Minni LI, Hefei City (CN)

(21) Appl. No.: 19/538,113

(22) Filed: Feb. 12, 2026

(30) Foreign Application Priority Data
Feb. 21, 2025 (CN) 202510197629.8

(51) Int. Cl. A61K 31/702; A61P 3/06; A61P 3/08

(52) U.S. Cl. CPC A61K 31/702; A61P 3/06; A61P 3/08

(57) ABSTRACT

Provided are a large-leaf yellow tea oligosaccharide with GLP-1 agonist activity and use thereof. This disclosure belongs to the technical field of food and medicine. The large-leaf yellow tea oligosaccharide is an oligosaccharide ELYP-3 with a molecular weight of 3.2×10³ Da. The large-leaf yellow tea oligosaccharide includes rhamnose, galacturonic acid, glucose, galactose, and arabinose in a molar ratio of 1:32.42:45.37:9.11:6.54. Beneficial effects: The large-leaf yellow tea oligosaccharide fragment obtained through glycosidase degradation in this disclosure exhibits significant blood glucose-lowering and weight-reducing activities. The large-leaf yellow tea oligosaccharide can be prepared by a simple, highly-specific, and eco-friendly process. Therefore, this disclosure provides a scientific basis and theoretical support for the development of natural GLP-1 agonists, and also offers promising application prospects for the use of functional oligosaccharides in the development of foods, drugs, or health products with blood sugar-regulating effects.

### CROSS REFERENCE TO RELATED APPLICATION

This patent application claims the benefit and priority of Chinese Patent Application No. 202510197629.8 filed with the China National Intellectual Property Administration on Feb. 21, 2025, the disclosure of which is incorporated by reference herein in its entirety as part of the present application.

### TECHNICAL FIELD

The present disclosure belongs to the field of food and medicine, and specifically relates to large-leaf yellow tea oligosaccharides with GLP-1 agonist activity and use thereof.

### BACKGROUND

Tea generally refers to a beverage made from the processed buds and leaves of tea plant (Camellia sinensis (L.) O. Kuntze), which is a perennial evergreen shrub of the genus Camellia in the family Theaceae. It is recognized as a healthy food that can promote wellness and prevent human diseases. In Chinese folk tradition, there are records of frequently consuming coarse, aged tea to treat diabetes. According to herbal studies, the pharmacologically active components in tea for treating diabetes are tea polysaccharides. Modern pharmacological studies have demonstrated that tea polysaccharides, as the major active components in tea, not only possess blood glucose-lowering effects but also exhibit a broad range of biological activities, including blood lipid reduction, blood coagulation inhibition, thrombosis prevention, immunity enhancement, cardiovascular protection, anticancer activity, oxidation resistance, and radiation protection. Tea polysaccharides are a class of acidic proteoglycans with complex structures and diverse functions. The rich and varied structural features of tea polysaccharides are the foundation for the various activities of tea polysaccharides. Due to the microheterogeneity in structures of tea polysaccharides, the diversity in monosaccharide compositions of tea polysaccharides, and the high flexibility and poor crystallizability of tea polysaccharides in a solution, the targeted research on tea polysaccharides remains significantly lagging. This is a key obstacle hindering the application of functional polysaccharides in clinical studies.

The core challenge in polysaccharide-targeting research lies in identifying sugar-recognition domains or binding sites. Therefore, degrading tea polysaccharides into oligosaccharides and studying active centers of polysaccharides at the oligosaccharide level offer a new approach to overcoming the bottleneck in understanding the structure-function relationship of tea polysaccharides. Currently, methods for degrading polysaccharides include physical degradation, chemical degradation, and enzymatic degradation. The physical degradation includes an ultrasonic process, a radiation process, a microwave process, a thermal treatment process, etc. The ultrasonic process is time-consuming and involves low efficiency, high energy consumption, and significant noise. The radiation process requires specialized devices and techniques, has high requirements on devices, and can easily cause hazards to operators. The microwave and thermal treatment processes may alter the physical and chemical properties of polysaccharides. The chemical degradation includes acid hydrolysis and oxidative degradation, which suffer from issues such as irregular reaction patterns, difficult process control, poor reproducibility, challenges in controlling compositions of degradation products, and environmental pollution. Compared to the physical degradation and the chemical degradation, the enzymatic degradation can cleave specific glycosidic bonds, and enables easily controllable reactions under mild conditions without generating by-products. Thus, the enzymatic degradation is a desirable degradation approach. Therefore, the enzymatic degradation of polysaccharides to produce functional oligosaccharides demonstrates promising prospects for industrialization.

Currently, there are relatively few research reports on the degradation of large-leaf yellow tea polysaccharides, and there are no reports on large-leaf yellow tea oligosaccharides with GLP-1 agonist activity.

### SUMMARY

The technical problem to be solved by the present disclosure is how to provide a large-leaf yellow tea oligosaccharide with glucagon-like peptide-1 (GLP-1) agonist activity.

The present disclosure solves the above technical problems through the following technical scheme.

A first aspect of the present disclosure provides a large-leaf yellow tea oligosaccharide with GLP-1 agonist activity, where the large-leaf yellow tea oligosaccharide is an oligosaccharide ELYP-3 with a molecular weight of 3.2×10³ Da, and the large-leaf yellow tea oligosaccharide includes rhamnose, galacturonic acid, glucose, galactose, and arabinose in a molar ratio of 1:32.42:45.37:9.11:6.54.

In some embodiments, the oligosaccharide ELYP-3 has a carbohydrate content of 93.59%.

In some embodiments, the oligosaccharide ELYP-3 includes a β-glycosidic bond and an α-glycosidic bond.

In some embodiments, the oligosaccharide ELYP-3 has a particle size predominantly ranging from 10 nm to 1,000 nm.

In some embodiments, the oligosaccharide ELYP-3 has an average particle size of 132.2 nm.

A second aspect of the present disclosure provides use of the large-leaf yellow tea oligosaccharide described above in any of the following:
(1) preparation of a product for promoting GLP-1 secretion by enteroendocrine cell line STC-1; and
(2) preparation of a product for preventing and/or treating diabetes by promoting GLP-1 secretion.

In some embodiments, in the (1) and the (2), the product is a drug.

A third aspect of the present disclosure provides a drug for preventing and/or treating diabetes, where an active ingredient of the drug includes the large-leaf yellow tea oligosaccharide described above.

In some embodiments, the drug further includes a pharmaceutically acceptable salt, including an organic salt or an inorganic salt.

In some embodiments, the drug further includes a pharmaceutically acceptable adjuvant.

In some embodiments, the pharmaceutically acceptable adjuvant is one or more selected from the group consisting of a diluent, an excipient, a filler, a binder, a wetting agent, a disintegrant, an absorption enhancer, a surfactant, an adsorption carrier, and a lubricant.

Embodiments of the present disclosure have the following beneficial effects:
1. The present disclosure provides a large-leaf yellow tea oligosaccharide ELYP-3 with GLP-1 agonist activity. The large-leaf yellow tea oligosaccharide includes rhamnose, galacturonic acid, glucose, galactose, and arabinose in a molar ratio of 1:32.42:45.37:9.11:6.54. Such a large-leaf yellow tea oligosaccharide fragment demonstrates significant efficacy in in vitro cell experiments. The large-leaf yellow tea oligosaccharide fragment may promote the secretion of GLP-1 by STC-1 cells and significantly up-regulate the expression levels of key genes involved in the synthesis and secretion of GLP-1.
2. The large-leaf yellow tea oligosaccharide ELYP-3 according to the present disclosure also demonstrates excellent efficacy in type II diabetes mouse models. The large-leaf yellow tea oligosaccharide fragment may significantly elevate the secretion levels of GLP-1 in the blood, ileum, and colon of mice, and also exhibit notable effects of lowering blood glucose, reducing blood lipids, decreasing lipid accumulation, and improving insulin resistance.
3. The large-leaf yellow tea oligosaccharide ELYP-3 according to the present disclosure lays a reliable substantial foundation for developing natural oligosaccharide-based hypoglycemic drugs with GLP-1 agonist activities or developing drugs with blood sugar-regulating effects.

### BRIEF DESCRIPTION OF THE DRAWINGS

FIG. 1 shows the influence of different large-leaf yellow tea oligosaccharide fragments in Example 1 of the present disclosure on GLP-1 secretion by enteroendocrine L cells;

FIGS. 2A-2B show an ultraviolet spectrum (FIG. 2A) and a Fourier transform infrared spectrum (FIG. 2B) of the large-leaf yellow tea oligosaccharide ELYP-3 in Example 2 of the present disclosure;

FIGS. 3A-3C show the molecular weight (FIG. 3A), the particle size (FIG. 3B), and the triple-helical structure spectrum (FIG. 3C) of the large-leaf yellow tea oligosaccharide ELYP-3 in Example 2 of the present disclosure;

FIGS. 4A-4B show scanning electron microscopy (SEM) images of the large-leaf yellow tea oligosaccharide ELYP-3 in Example 2 of the present disclosure, where FIG. 4A: LYP and FIG. 4B: ELYP-3;

FIGS. 5A-5B show monosaccharide composition profiles of the large-leaf yellow tea oligosaccharide ELYP-3 in Example 2 of the present disclosure, where FIG. 5A: standard and FIG. 5B: ELYP-3;

FIGS. 6A-6I show the influence of the large-leaf yellow tea oligosaccharide ELYP-3 on promoting the synthesis and secretion of GLP-1 by enteroendocrine L cells (STC-1) in Example 3 of the present disclosure;

FIGS. 7A-7B show the influence of the large-leaf yellow tea oligosaccharide ELYP-3 on the concentration of GLP-1 in portal vein plasma when the large-leaf yellow tea oligosaccharide is directly administered to the jejunum (FIG. 7A) or ileum (FIG. 7B) of rats in Example 4 of the present disclosure;

FIGS. 8A-8I show the influence of the large-leaf yellow tea oligosaccharide ELYP-3 on the plasma GLP-1 level (FIG. 8A) and the synthesis and secretion of GLP-1 in the ileum (FIG. 8B to FIG. 8I) of type II diabetes mice in Example 4 of the present disclosure; and

FIGS. 9A-9H show the influence of the large-leaf yellow tea oligosaccharide ELYP-3 on the blood glucose level (FIG. 9A), insulin resistance (FIG. 9B to FIG. 9D), and a blood lipid level (FIG. 9E to FIG. 9H) in type II diabetes mice in Example 5 of the present disclosure.

### DETAILED DESCRIPTION OF THE EMBODIMENTS

In order to make the objectives, technical solutions, and advantages of the embodiments of the present disclosure clear, the technical solutions in the embodiments of the present disclosure will be clearly and completely described below with reference to the examples of the present disclosure. Apparently, the described examples are some rather than all of the embodiments of the present disclosure. All other embodiments obtained by those of ordinary skill in the art based on the embodiments of the present disclosure without creative efforts shall fall within the scope of protection of the present disclosure.

The experimental materials and reagents used in the following examples are all commercially available, unless otherwise specified.

If specific techniques or conditions are not indicated in the examples, a process shall be conducted in accordance with the techniques or conditions described in literatures in the art or in accordance with a product specification. Unless otherwise specified, three or more replicates are set for each of the quantitative tests in the following examples, and results are averaged.

### Example 1: Preparation of Large-Leaf Yellow Tea Oligosaccharide Fragments

(1) Preparation of large-leaf yellow tea polysaccharides: large-leaf yellow tea was oven-dried and crushed to produce large-leaf yellow tea powder. An appropriate amount of the large-leaf yellow tea powder was mixed with 95% ethanol at a solid-to-liquid ratio of 1:20 g/mL to produce a mixture. The mixture was stirred at 25 °C and 50 rpm/min for 24 h to remove fat and pigment impurities and subjected to suction filtration to produce a filter residue. The filter residue was subjected to hot water extraction at 90 °C under stirring for 2.5 h at a solid-to-liquid ratio of 1:20 g/mL. Filtration was conducted to produce a filtrate. Absolute ethanol (final ethanol concentration: 80%) was added in a volume 4 times the volume of the filtrate to the filtrate to produce a mixed solution. The mixed solution was allowed to stand at 4 °C for 12 h and then centrifuged to produce a precipitate. Proteins in the precipitate were removed by the Sevage method. The resulting protein-free polysaccharide solution was concentrated by rotary evaporation, dialyzed, lyophilized, and further purified using a N, N-Diethylethanolamine (DEAE) cellulose DE-52 anion exchange chromatography column and a Sephadex G-100 gel filtration chromatography column to produce a homogeneous large-leaf yellow tea polysaccharide fraction LYP.

(2) Preparation of large-leaf yellow tea oligosaccharide fragments: The purified large-leaf yellow tea polysaccharide was dissolved in a 50 mM acetic acid-sodium acetate buffer with a pH of 4.0 to prepare a 1 mg/mL large-leaf yellow tea polysaccharide solution. Galacturonase was added at 200 U/mg, and enzymatic hydrolysis was conducted at 50 °C for 12 h. After the enzymatic hydrolysis was completed, boiling was conducted to enable enzyme deactivation to produce a reaction solution. The reaction solution was centrifuged (5,700 g, 10 min) to produce a supernatant. The supernatant was collected, subjected to desalting and impurity removal by an ultrafiltration membrane with a molecular weight cut-off of 0.5 KDa, and then allowed to pass through membrane modules of 1 KDa, 3 KDa, and 5 KDa sequentially to produce retentates and flow-through. The retentates and the flow-through were each collected, concentrated, and lyophilized to produce oligosaccharide components with Mw of 0.5 KDa to 1 KDa, 1 KDa to 3 KDa, and 3 KDa to 5 KDa, which were designated as ELYP-1, ELYP-2, and ELYP-3, respectively.

(3) Screening of the large-leaf yellow tea oligosaccharide fragments for the activity of promoting GLP-1 secretion by enteroendocrine L cells: The large-leaf yellow tea oligosaccharide fragments ELYP-1, ELYP-2, and ELYP-3 with different degrees of polymerization were each administered to enteroendocrine L cells (STC-1) to evaluate the activity of promoting GLP-1 secretion. STC-1 cells in a prominent growth status were collected, inoculated into a 48-well plate (cell density: 1×10⁶ cells/mL), and cultured in a 5% CO₂ incubator for 24 h. The medium was removed. ELYP-1, ELYP-2, and ELYP-3 solutions were each added at a final concentration of 100 μg/mL , and incubation was continued for 2 h. A complete medium was added to set a blank control group (control). The homogeneous large-leaf yellow tea polysaccharide was added to set an LYP group. After the incubation was completed, a resulting culture was collected and centrifuged (400 g, 4 °C, 15 min) to produce a supernatant. The GLP-1 content in the supernatant was determined.

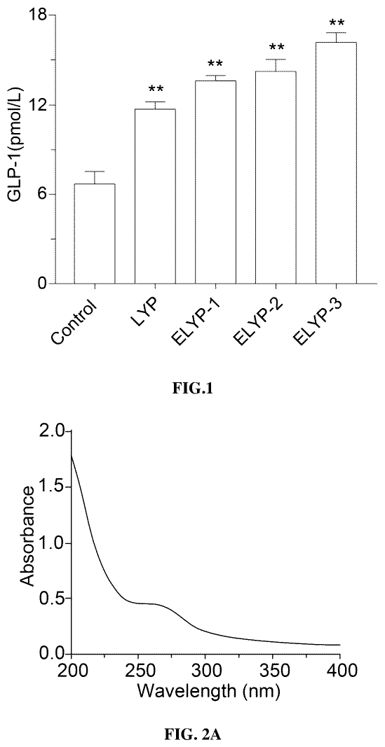

Results are shown in FIG. 1. Compared with the control, the LYP and the oligosaccharide fragments ELYP-1, ELYP-2, and ELYP-3 with different degrees of polymerization obtained through galacturonase degradation significantly increased the level of GLP-1 secreted by STC-1 cells. GLP-1 levels in the LYP and oligosaccharide fragment groups were 1.76, 2.05, 2.14, and 2.44 times that in the control group, respectively. It can be seen that ELYP-3 exhibited the highest activity in promoting GLP-1 secretion by STC-1 cells. The GLP-1 secretion level induced by ELYP-3 in STC-1 cells was 1.39 times that induced by LYP, 1.19 times that induced by ELYP-1, and 1.14 times that induced by ELYP-2. Therefore, ELYP-3 is the optimal active fragment for promoting GLP-1 secretion by enteroendocrine cells.

### Example 2: Purification and Structural Characterization for the Large-Leaf Yellow Tea Oligosaccharide Fragment ELYP-3

(1) Purification of the large-leaf yellow tea oligosaccharide fragment The component ELYP-3 in Example 1 was dissolved in double-distilled water and loaded onto a Bio-Gel P-2 polyacrylamide gel column at a concentration of 40 mg/mL. Elution was conducted with deionized water at a flow rate of 10 mL/h. The polysaccharide content in the eluate was detected by the phenol-sulfuric acid method. The sugar-containing eluate was collected, concentrated under vacuum, and lyophilized to produce a purified large-leaf yellow tea oligosaccharide ELYP-3.

(2) Determination of the carbohydrate content in the large-leaf yellow tea oligosaccharide ELYP-3:

The carbohydrate content in ELYP-3 obtained in the (1) was determined by the phenol-sulfuric acid method. As measured by spectrophotometry at 490 nm, the carbohydrate content in ELYP-3 was 93.59%.

(3) Ultraviolet spectroscopy of ELYP-3:

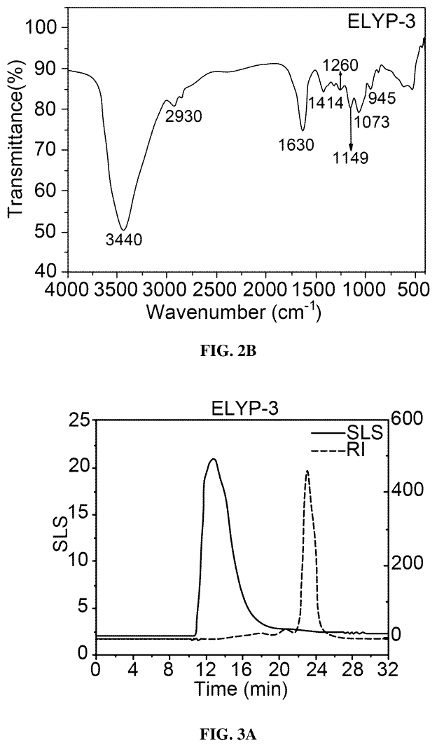

ELYP-3 was scanned across a wavelength range of 190 nm to 400 nm using a full-wavelength scanning microplate reader. Results are shown in FIG. 2A. ELYP-3 exhibited no absorption peak at 260 nm or 280 nm in the scan spectrum, indicating that the purified oligosaccharide resulting from enzymatic hydrolysis includes almost no pigments, proteins, or nucleic acids.

(4) Infrared spectroscopy of ELYP-3:

Characteristic functional groups of ELYP-3 were analyzed by scanning of a Nicolette iS50 Fourier transform infrared spectrometer. Results are shown in FIG. 2B. ELYP-3 exhibited typical characteristic sugar absorption peaks, including: a signal peak at 3,440 cm⁻¹, which is attributed to an intramolecular or intermolecular O—H stretching vibration in sugar molecules; a signal peak at 2,930 cm⁻¹, which is attributed to a stretching vibration of C—H; signal peaks at 1,630 cm⁻¹ and 1,414 cm⁻¹, which are attributed to a stretching vibration of carbonyl (C═O) in COOH and a bending vibration of C—H, respectively; a signal peak at 1,149 cm⁻¹, which corresponds to stretching vibrations of C—O—C and C—O—H in a pyranose ring; and a signal peak at 946 cm⁻¹, which indicates the presence of both a β-glycosidic bond and an α-glycosidic bond in ELYP-3.

(5) Determination of the molecular weight of the large-leaf yellow tea oligosaccharide ELYP-3:

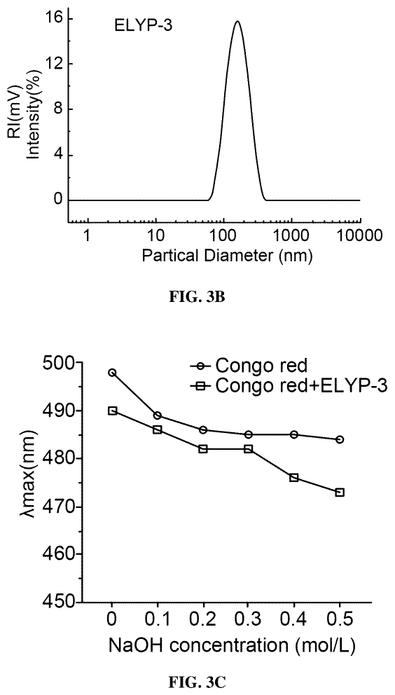

The molecular weight distribution of ELYP-3 was determined using high-performance gel permeation chromatography coupled with refractive index (RI) and multi-angle laser light scattering detection. Results are shown in FIG. 3A. The molecular weight of ELYP-3 is 3.2×10³ Da. Detection conditions: Waters Arc high performance liquid chromatography (HPLC) system; Brookhaven BI-MwA multi-angle laser light scattering detector; Waters 2414 RI detector; TSK G 2500PWXL analytical column (7.8 mm×300 mm); mobile phase: 0.1 M NaNO₃ solution; flow rate: 0.6 mL/min; column temperature: 35 °C; and injection volume: 100 μL.

(6) Determination of the particle size of the large-leaf yellow tea oligosaccharide ELYP-3:

The particle size of ELYP-3 was determined by a Zetasizer Pro nanoparticle size analyzer. 1 mg/mL of ELYP-3 solution was prepared, filtered through a 0.22 μm aqueous membrane, and tested for the particle size under conditions of 25 °C, a wavelength of 633 nm, and a scattering angle of 90°. As shown in FIG. 3B, ELYP-3 had a particle size primarily concentrated in a range of 10 nm to 1,000 nm and an average particle size of 132.2 nm.

(7) Determination of the triple-helical structure in the large-leaf yellow tea oligosaccharide ELYP-3:

Whether there was a triple-helical structure in ELYP-3 was determined through Congo red binding assay. A 2 mg/mL ELYP-3 solution was prepared and thoroughly mixed with a Congo red solution according to 1:1 v/v. NaOH was added at different final concentrations (0 mol/L to 0.5 mol/L). A mixed solution without the ELYP-3 solution was taken as a control. Resulting mixed systems were each allowed to stand at room temperature for 10 min. The maximum absorption wavelength of the Congo red-large-leaf yellow tea oligosaccharide complex was determined by a full-wavelength ultraviolet spectrophotometer. As shown in FIG. 3C, with the increase of the NaOH concentration, the maximum absorption wavelength of the Congo red-ELYP-3 complex decreased and eventually stabilized, indicating that ELYP-3 does not possess a triple-helical structure.

(8) Microstructural analysis of the large-leaf yellow tea oligosaccharide ELYP-3:

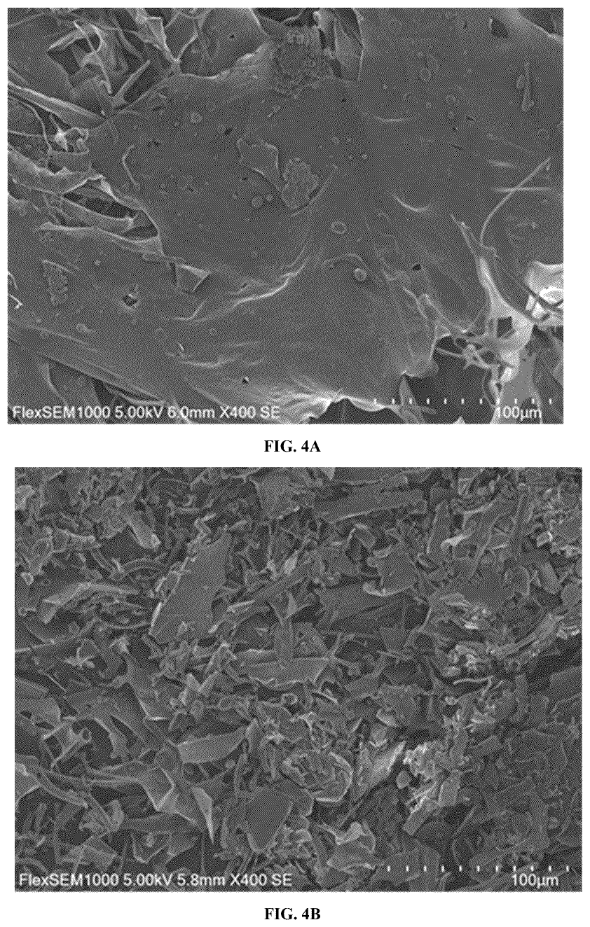

The apparent morphology of ELYP-3 was analyzed by a Hitachi SU1000 scanning electron microscope. 2 mg of dried ELYP-3 was weighed and adhered to a metal stub with a conductive adhesive. A conductive film was coated on the surface of the sample, and then, the apparent morphology was observed. Results are shown in FIG. 4B. The surface of ELYP-3 presents fragmented, irregular particulate segments.

(9) Monosaccharide composition analysis of the large-leaf yellow tea oligosaccharide ELYP-3:

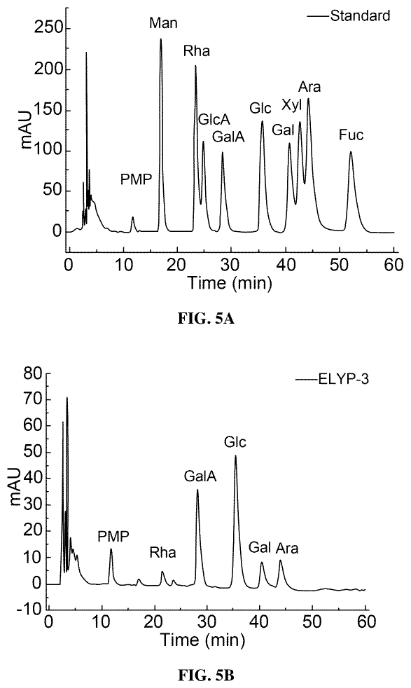

The monosaccharide composition of ELYP-3 was determined using HPLC with pre-column 1-phenyl-3-methyl-5-pyrazolone (PMP) derivatization. 5 mg of dried ELYP-3 was weighed and placed in a 10 mL ampoule bottle. 3 mL of a 3 M hydrochloric acid solution was added to the ampoule bottle. The ampoule bottle was sealed. Hydrolysis was conducted at 110 °C for 1 h, and then cooling was conducted. The pH of a resulting reaction solution was adjusted to 7.0 with a 3.0 mol/L sodium hydroxide solution to produce an oligosaccharide hydrolysate. 100 μL of a 0.6 M NaOH solution and 100 μL of a solution of 0.6 M PMP in methanol were added to the oligosaccharide hydrolysate. The reaction was conducted at 70 °C for 100 min. After the reaction was completed, 50 μL of a 0.3 M HCl solution was added. Rotary evaporation was conducted at 50 °C to dryness. Washing was conducted with chloroform three times. A resulting product was collected through filtration and tested by HPLC. Results are shown in FIG. 5. In contrast to chromatograms of monosaccharide standards, ELYP-3 primarily includes rhamnose, galacturonic acid, glucose, galactose, and arabinose in a molar ratio of 1:32.42:45.37:9.11:6.54.

Detection conditions: Waters Arc HPLC system; Waters-C18 column (250 mm×4.6 mm); ultraviolet detector; column temperature: 30 °C; mobile phase: 0.1 mol/L phosphate buffered saline (PBS) (pH 6.7): acetonitrile=83:17 (v/v); flow rate: 1.0 mL/min; and detection wavelength: 245 nm.

### Example 3: Influence of the Large-Leaf Yellow Tea Oligosaccharide Fragment on the Synthesis and Secretion of GLP-1 by Enteroendocrine L Cells in Vitro

With enteroendocrine L cell line STC-1 as a model, the promotion of the ELYP-3 prepared in Example 1 on the synthesis and secretion of GLP-1 by enteroendocrine L cells was investigated. STC-1 cells in the logarithmic growth phase were collected and adjusted to a concentration of 1×10⁶ cells/mL, then inoculated into a 6-well cell culture plate, and adherently cultured for 24 h in an incubator at 37 °C and 5% CO₂. ELYP-3 was added at final concentrations of 25μg/mL and 100 μg/mL , and incubation was conducted for 2 h. A complete medium was set as a blank control group. After the incubation was completed, cells and a cell supernatant were collected from each group mRNA expression levels of key genes (gcg, pcsk1, α-gustducin, plcb2, gipr, glp1r, and tlr2/3) involved in the synthesis of a GLP-1 precursor, the processing of the GLP-1 precursor into a mature form, and the extracellular release of GLP-1 in the cells were detected by reverse transcription quantitative polymerase chain reaction (RT-qPCR). The content of GLP-1 in the cell supernatant was measured by enzyme-linked immunosorbent assay (ELISA).

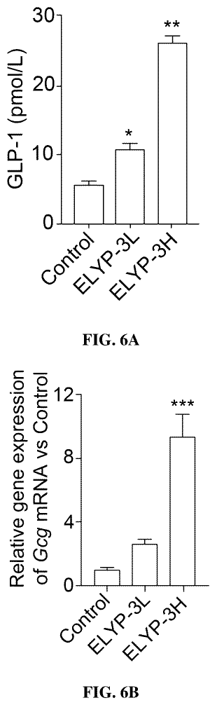

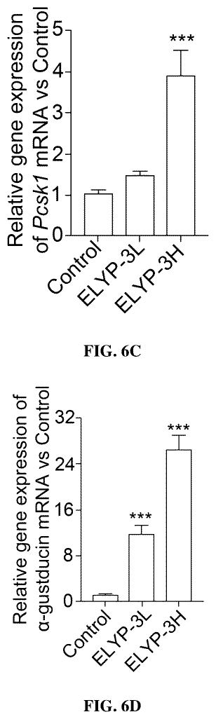

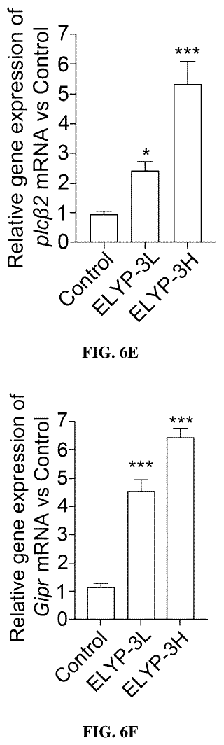

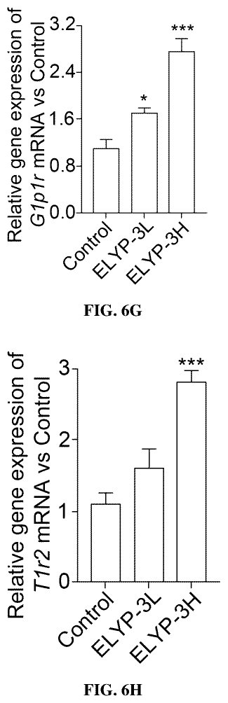

Results are shown in FIGS. 6A-6I. ELYP-3 significantly promoted the efficient secretion of GLP-1 by enteroendocrine cell line STC-1 and up-regulated the mRNA expression levels of key genes involved in the synthesis of a GLP-1 precursor (gcg and pcsk1), the processing of the GLP-1 precursor into a mature form (a-gustducin and plcb2), and the extracellular release of GLP-1 (glp1r and tlr2/3), with a dose-dependent effect. The above results indicate that, when acting on STC-1 cells, ELYP-3 can not only effectively increase the intracellular synthesis of GLP-1 but also significantly promote the release of GLP-1 from the intracellular space to the extracellular space.

### Example 4: Influence of the Large-Leaf Yellow Tea Oligosaccharide Fragment on the Synthesis and Secretion of GLP-1 in Normal Mice and T2DM Mice

(1) In situ detection of GLP-1

Five-week-old male SD rats were selected and acclimatized in a specific-pathogen free (SPF)-grade animal room for one week under the following conditions: temperature: 23+2 °C, relative humidity: 50% to 60%, and 12 h light/12 h dark cycle. Then, the male SD rats were randomly divided into the following three groups (n=6): a control group (in situ administration of normal saline), a low-concentration ELYP-3 in situ administration group (ELYP-3L: 0.5 g/kg), and a high-concentration ELYP-3 in situ administration group (ELYP-3H: 2 g/kg). Rats in each group were placed on a surgical table and anesthetized. The abdominal cavity was opened, and a catheter was inserted into a hepatic portal vein. The jejunum or ileum was then ligated. The drug was administered directly to the ligated jejunum or ileum. At 15 min, 30 min, 60 min, 90 min, and 120 min after the administration, blood was collected from the hepatic portal vein using a syringe including ethylenediaminetetraacetic acid disodium salt (EDTA-2Na) (1 mg/mL), aprotinin (500 kIU/mL), and a DPP-IV inhibitor (100 mM), and centrifuged at 3,500 rpm and 4 °C or 15 min to collect plasma. Secretion levels of plasma GLP-1 at different time points after administration were determined.

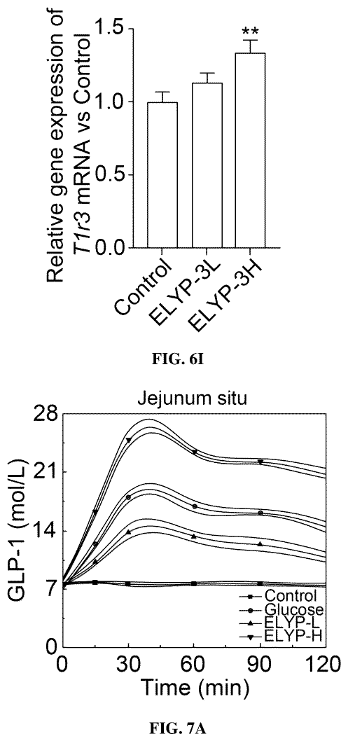

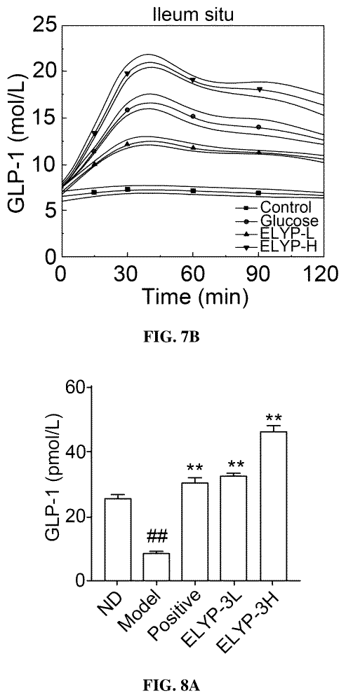

To further investigate whether ELYP-3 can directly act on enteroendocrine L cells in vivo to promote GLP-1 secretion, an in situ intestinal administration experiment of ELYP-3 in vivo was conducted to assess plasma GLP-1 levels after the in situ direct administration of ELYP-3 to the jejunum or ileum. Results are shown in FIG. 7. Compared to the control group, the plasma GLP-1 level increased rapidly within 15 min after the direct administration of ELYP-3 to the jejunum or ileum, and peaked at 30 min. After 30 min, the plasma GLP-1 level gradually plateaued. The results indicate that ELYP-3 can directly act on enteroendocrine L cells in vivo to promote GLP-1 secretion.

(2) Determination of GLP-1 synthesis and secretion levels in Type 2 Diabetes Mellitus (T2DM) mice

T2DM mouse model establishment and drug intervention: Four-week-old male C57/6J mice were selected and acclimatized for one week in an SPF-grade animal room under the following conditions: temperature: 23±2 °C, relative humidity: 50% to 60%, and 12 h light/12 h dark cycle. The male C57/6J mice were randomly divided into the following groups with 12 mice per group: a normal group (ND: feeding of a standard diet+intragastric administration of normal saline), a high-fat diet (HFD) model group (HFD: feeding of HFD+intragastric administration of normal saline), a positive group (positive: feeding of HFD+intragastric administration of metformin at 50 mg/kg/d), a low-dose ELYP-3 group (ELYP-3L: feeding of HFD+intragastric administration of ELYP-3 at 50 mg/kg/d), and a high-dose ELYP-3 group (ELYP-3H: feeding of HFD+intragastric administration of ELYP-3 at 200 mg/kg/d). After 8 weeks of high-fat feeding, mice were each intraperitoneally injected with streptozotocin (STZ) once at 100 mg/kg of body weight (BW). The fasting blood glucose (FBG) level was determined at 72 h after STZ injection. Mice with an FBG level of 11.1 mmol/L or more were selected as diabetic model mice. The corresponding intervention substance was intragastrically administered consecutively until the end of the experiment at week 12. Mice were fasted overnight and sacrificed with CO₂. Cardiac blood was collected. Colon and ileum tissues were collected, frozen in liquid nitrogen, and stored at 80 °C for later use.

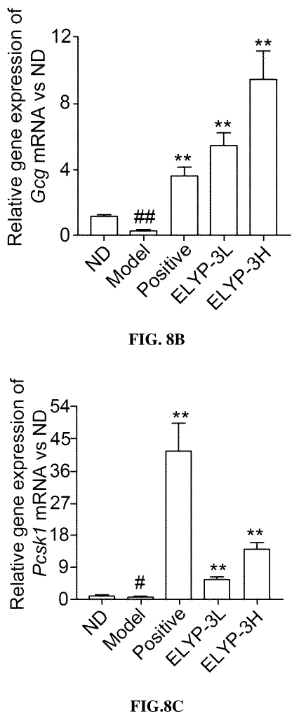

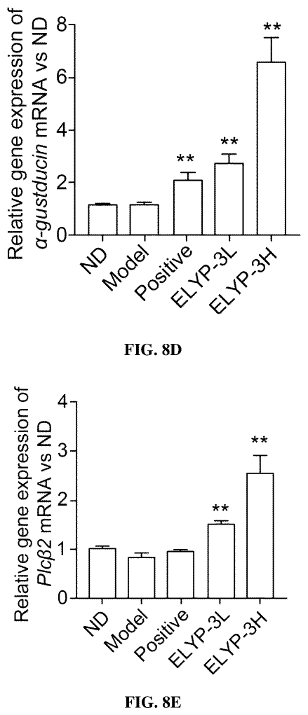

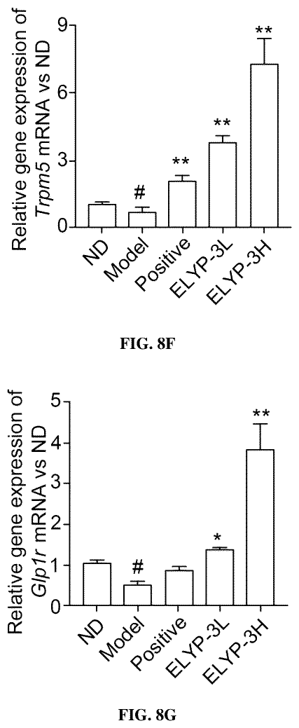

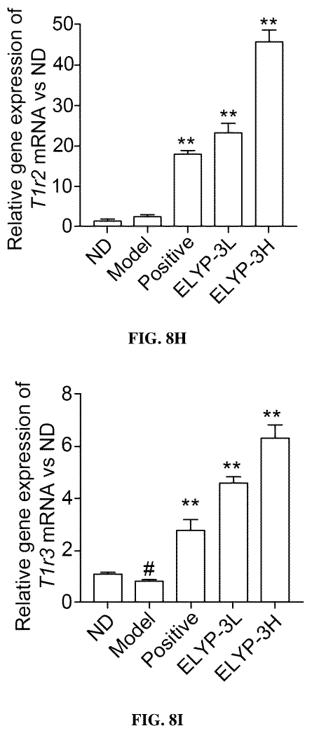

T2DM model mice were constructed through HFD feeding and STZ injection to investigate the influence of the large-leaf yellow tea oligosaccharide of the present disclosure on the synthesis and secretion of GLP-1 in the T2DM model mice. Results are shown in FIG. 8A. Compared with the ND group, the plasma GLP-1 level in the T2DM model mice significantly decreased. The intervention with ELYP-3 at different doses significantly increased the plasma GLP-1 level in the T2DM model mice. To further investigate the influence of ELYP-3 on the synthesis and secretion of GLP-1 by enteroendocrine L cells in the T2DM model mice, mRNA expression levels of key genes (gcg, pcsk1, a-gustducin, plcb2, trpm5, glp1r, and tlr2/3) involved in the synthesis of a GLP-1 precursor, the processing of the GLP-1 precursor into a mature form, and the extracellular release of GLP-1 in the ileum tissue were detected by RT-qPCR. Results are shown in FIG. 8B to FIG. 8I. The ELYP-3 intervention significantly up-regulated the mRNA expression levels of key genes involved in the synthesis of GLP-1 precursor (gcg and pcsk1), the processing of the GLP-1 precursor into mature form (a-gustducin and plcb2), and the extracellular release of GLP-1 (trpm5, glpIr, and tlr2/3) in the ileum of the T2DM model mice, with a dose-dependent effect. The above results indicate that ELYP-3 can activate the GLP-1 receptor in the T2DM model mice, which also can not only effectively increase the intracellular synthesis of GLP-1 but also significantly promote the release of GLP-1 from the intracellular space to the extracellular space.

### Example 5: Improvement Effects of the Large-Leaf Yellow Tea Oligosaccharide Fragment on a Blood Glucose Level, a Blood Lipid Level, and Insulin Resistance in T2DM Mice

During an experiment, FBG levels in the T2DM mice were monitored weekly using ACCU-CHEK Performa. At the end of the experiment, plasma lipid indexes including total cholesterol (TC), triglyceride (TG), low-density lipoprotein cholesterol (LDL-c), and high-density lipoprotein cholesterol (HDL-c) in T2DM mice of each group were detected by biochemical assay kits, and the fasting insulin (FINS) level in the T2DM mice was determined by an ELISA kit. The insulin resistance index (HOMA-IR) and β-cell function index (HOMA-β) were calculated according to the following formulas, respectively: HOMA-IR=FBG (mmol/L)×FINS (mU/L)/22.5 and HOMA-β=[20×FINS (mU/L)]/[FBG (mmol/L)-3.5]. Improvement effects of the large-leaf yellow tea oligosaccharide of the present disclosure on a blood glucose level, a blood lipid level, and insulin resistance in the T2DM mice were investigated.

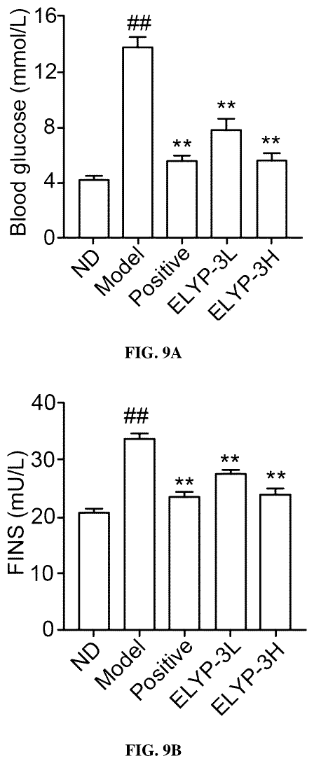

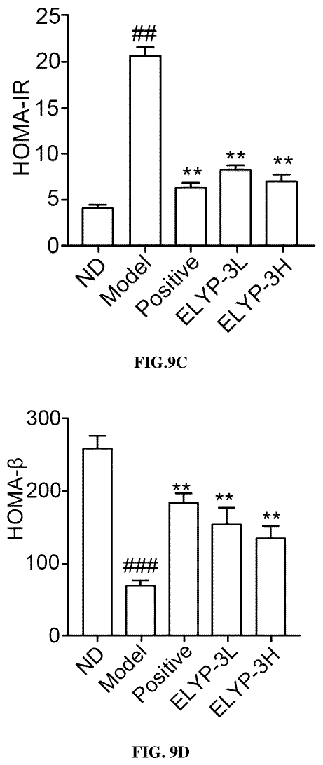

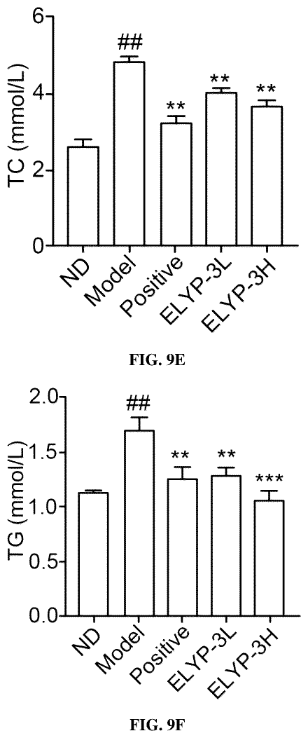

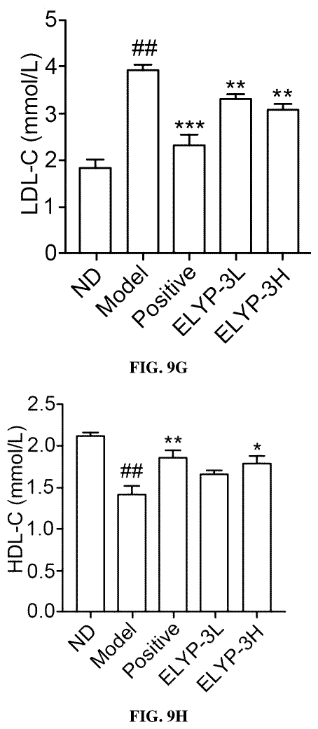

As shown in FIG. 9A to FIG. 9B, the T2DM model group exhibited significantly higher FBG and FINS levels than the ND group. According to these results, the T2DM mice exhibited characteristic hyperglycemia and insulin resistance. After the intervention with ELYP-3 at different doses, both FBG and FINS levels in the T2DM mice were significantly reduced, indicating that the ELYP-3 intervention has an auxiliary hypoglycemic effect. Calculation results of the insulin resistance index and -cell function index for each group of mice are shown in FIG. 9C to FIG. 9D. The ELYP-3 intervention significantly decreased HOMA-IR and increased HOMA-β in the T2DM mice. It indicates that ELYP-3 can markedly ameliorate insulin resistance and enhance insulin sensitivity in the T2DM mice.

The blood lipid level in the T2DM mice was characterized by measuring the TC, TG, HDL-c, and LDL-c in the plasma of the T2DM mice. Results are shown in FIG. 9E to FIG. 9H. The intervention with ELYP-3 at different doses reduced the TC, TG, and LDL-c levels and increased the HDL-c level in the plasma of the T2DM mice. This indicates that the ELYP-3 intervention can significantly ameliorate hyperlipidemia in the T2DM mice and exert an auxiliary lipid-lowering effect.

The above examples are intended merely to describe the technical solutions of the present disclosure, and are not intended to limit the present disclosure. Although the present disclosure is described in detail with reference to the above examples, it should be understood that those of ordinary skill in the art can still modify the technical solutions described in the above examples, or make equivalent substitutions to some technical features therein. These modifications or substitutions do not make the essence of the corresponding technical solutions depart from the spirit and scope of the technical solutions of the examples of the present disclosure.

1. A large-leaf yellow tea oligosaccharide with glucagon-like peptide-1 (GLP-1) agonist activity, wherein the large-leaf yellow tea oligosaccharide is an oligosaccharide ELYP-3 with a molecular weight of 3.2×10³ Da, and the large-leaf yellow tea oligosaccharide comprises rhamnose, galacturonic acid, glucose, galactose, and arabinose in a molar ratio of 1:32.42:45.37:9.11:6.54.

2. The large-leaf yellow tea oligosaccharide with GLP-1 agonist activity according to claim 1, wherein the oligosaccharide ELYP-3 has a carbohydrate content 93.59%.

3. The large-leaf yellow tea oligosaccharide with GLP-1 agonist activity according to claim 1, wherein the oligosaccharide ELYP-3 comprises a β-glycosidic bond and an α-glycosidic bond.

4. The large-leaf yellow tea oligosaccharide with GLP-1 agonist activity according to claim 1, wherein the oligosaccharide ELYP-3 has a particle size predominantly ranging from 10 nm to 1,000 nm.

5. The large-leaf yellow tea oligosaccharide with GLP-1 agonist activity according to claim 1, wherein the oligosaccharide ELYP-3 has an average particle size of 132.2 nm.

6. Use-A method for preventing and/or treating diabetes by promoting GLP-1 secretion, comprising administering to a subject in need thereof a product comprising a therapeutically effective amount of the large-leaf yellow tea oligosaccharide according to claim 1,
wherein the product comprises a food, a drug, or a health product.

7. A drug for preventing and/or treating diabetes, wherein an active ingredient of the drug comprises the large-leaf yellow tea oligosaccharide according to claim 1.

8. The drug for preventing and/or treating diabetes according to claim 7, wherein the drug further comprises a pharmaceutically acceptable salt, comprising an organic salt or an inorganic salt.

9. The drug for preventing and/or treating diabetes according to claim 7, wherein the drug further comprises a pharmaceutically acceptable adjuvant.

10. The drug for preventing and/or treating diabetes according to claim 9, wherein the pharmaceutically acceptable adjuvant is one or more selected from the group consisting of a diluent, an excipient, a filler, a binder, a wetting agent, a disintegrant, an absorption enhancer, a surfactant, an adsorption carrier, and a lubricant.

11. The method according to claim 6, wherein the oligosaccharide ELYP-3 has a carbohydrate content 93.59%.

12. The method according to claim 6, wherein the oligosaccharide ELYP-3 comprises a β-glycosidic bond and an α-glycosidic bond.

13. The method according to claim 6, wherein the oligosaccharide ELYP-3 has a particle size predominantly ranging from 10 nm to 1,000 nm.

14. The method according to claim 6, wherein the oligosaccharide ELYP-3 has an average particle size of 132.2 nm.

15. The drug according to claim 7, wherein the oligosaccharide ELYP-3 has a carbohydrate content 93.59%.

16. The drug according to claim 7, wherein the oligosaccharide ELYP-3 comprises a β-glycosidic bond and an α-glycosidic bond.

17. The drug according to claim 7, wherein the oligosaccharide ELYP-3 has a particle size predominantly ranging from 10 nm to 1,000 nm.

18. The drug according to claim 7, wherein the oligosaccharide ELYP-3 has an average particle size of 132.2 nm.

19. A method for promoting GLP-1 secretion by an enteroendocrine cell STC-1, comprising administering to a subject in need thereof a product comprising a therapeutically effective amount of the large-leaf yellow tea oligosaccharide according to claim 1.

20. The method according to claim 19, wherein the oligosaccharide ELYP-3 has an average particle size of 132.2 nm.
